(19) United States
(12) Patent Application Publication
(10) Pub. No.: US 2009/0080669 A1
(43) Pub. Date: Mar. 26, 2009

(54) NOISE CANCEL METHOD, NOISE CANCEL TYPE AMPLIFYING CIRCUIT, AND RECEIVING CIRCUIT AND ELECTRONIC DEVICE INCLUDING NOISE CANCEL TYPE AMPLIFYING CIRCUIT

(75) Inventors: Koichi HATANAKA, Matsumoto (JP); Tadashi AIZAWA, Matsumoto (JP)

Correspondence Address: GLOBAL IP COUNSELORS, LLP, 1233 20TH STREET, NW, SUITE 700, WASHINGTON, DC, 20036-2680, US

(73) Assignee: SEIKO EPSON CORPORATION, Tokyo (JP)

(21) Appl. No.: 12/209,484

(22) Filed: Sep. 12, 2008

(30) Foreign Application Priority Data
Sep. 26, 2007 (JP) 2007-248582
Jul. 3, 2008 (JP) 2008-174258

(51) Int. Cl. A61F 11/06

(52) U.S. Cl. 381/71.1

(57) ABSTRACT

A noise cancel method includes: generating a cancel signal which cancels a noise signal based on the noise signal generated from a receiving unit for receiving a wireless signal; generating an amplification signal produced by amplifying a reception signal inputted to the receiving unit; and generating an addition signal produced by adding the amplification signal and the cancel signal.

### BACKGROUND

1. Technical Field

The present invention relates to a noise cancel method, a noise cancel type amplifying circuit, and a receiving circuit and an electronic device including the noise cancel type amplifying circuit.

2. Related Art

According to an electronic device containing a receiving circuit, alternating current signals are generated due to changes of electromagnetic field produced by circuit operation of an electronic circuit disposed in the vicinity of the receiving circuit. In this case, there is a possibility that the generated alternating current signals reach the receiving circuit to be mixed into reception signals as interference waves. This phenomenon becomes a great factor for causing signal deterioration, and thus various technologies for removing interference signals superimposed on reception signals have been developed.

JP-A-2006-145315 discloses an example of these technologies. According to this disclosure, signals for canceling generation signals generated from an electronic circuit (hereinafter referred to as “cancel signals”) are produced by inverting phase of the generation signals using a delay line to remove interference waves.

Signals received by a receiving unit such as an antenna are weak signals, and thus the signal level generally needs to be raised by amplification before signal processing. According to the technology disclosed in JP-A-2006-145315, however, a contact of the delay line is provided at an intermediate position on a signal line for connecting the antenna and a signal processing system before the amplification step to add the cancel signal to the weak signal immediately after signal reception.

According to this structure, the signal level of the cancel signal to be added needs to be accurately controlled such that interference wave component mixed in the weak reception signal can be appropriately canceled. When the signal level of the cancel signal is inaccurate, the interference wave component rather increases by generation of excessive cancel signal which exceeds the signal level sufficient for canceling the interference wave component. As a result, receiving sensitivity decreases.

Moreover, noise in a frequency band of a desired signal cannot be removed by band-pass filter by related-art method. According to the band-pass filter, noise at the same frequency cannot be distinguished from the desired signal, and thus is allowed to pass through the band as a signal within the frequency band without attenuation. The noise in the frequency band deteriorates receiving capability of a receiver even when the noise has extremely weak power.

### SUMMARY

It is an advantage of some aspects of the invention to provide a noise cancel method, a noise cancel type amplifying circuit, and a receiving circuit and an electronic device including the noise cancel type amplifying circuit, which can solve at least a part of the above problems. This advantage can be provided by the following examples and applications.

A noise cancel method according to a first aspect of the invention includes: generating a cancel signal which cancels a noise signal based on the noise signal generated from a receiving unit for receiving a wireless signal; generating an amplification signal produced by amplifying a reception signal inputted to the receiving unit; and generating an addition signal produced by adding the amplification signal and the cancel signal.

According to this structure, the noise signal affecting the quality of the reception signal can be effectively removed. Thus, the receiving capability improves.

It is preferable to further include performing amplitude control of the noise signal based on the addition signal at the time of generation of the cancel signal in the noise cancel method.

According to this structure, the noise signal affecting the quality of the reception signal can be effectively removed. Thus, the receiving capability improves.

It is preferable to further include performing amplification or attenuation of the noise signal based on the addition signal at the time of generation of the cancel signal in the noise cancel method.

According to this structure, the noise signal affecting the quality of the reception signal can be effectively removed. Thus, the receiving capability improves.

It is preferable that the reception signal is a positioning satellite signal received from a positioning satellite in the noise cancel method.

According to this structure, the noise signal affecting the quality of the positioning satellite signal received from the positioning satellite can be effectively removed. Thus, the receiving capability improves.

A noise cancel type amplifying circuit according to a second aspect of the invention includes: a first pin through which a reception signal is inputted; a second pin through which a noise signal affecting the reception signal is inputted; a low-noise amplifying circuit connected with the first pin to input the reception signal and output an amplification signal; a cancel signal generating unit connected with the second pin to generate a cancel signal for canceling the noise signal based on the noise signal inputted through the second pin; and an adder which outputs an addition signal produced by adding the amplification signal and the cancel signal through a third pin.

According to this structure, the noise signal affecting the quality of the reception signal can be effectively removed. Thus, the receiving capability improves.

It is preferable that the cancel signal generating unit has a phase shift unit for outputting a phase shift signal produced by arbitrarily shifting the phase of the noise signal, and an amplitude control unit for controlling the amplitude of the phase shift signal in the noise cancel type amplifying circuit.

According to this structure, the noise signal affecting the quality of the reception signal can be effectively removed. Thus, the receiving capability improves.

It is preferable that the amplitude control unit includes at least either an amplifying unit or an attenuating unit in the noise cancel type amplifying circuit.

According to this structure, the noise signal affecting the quality of the reception signal can be effectively removed. Thus, the receiving capability improves.

It is preferable that at least either the amplification rate of the amplifying unit or the attenuation rate of the attenuating unit is determined based on the addition signal in the noise cancel type amplifying circuit.

According to this structure, the noise signal affecting the quality of the reception signal can be effectively removed. Thus, the receiving capability improves.

It is preferable that the reception signal is a positioning satellite signal received from a positioning satellite, and that the noise signal is a signal generated from a circuit disposed in the vicinity of a receiving unit for receiving the receiving signal in the noise cancel type amplifying circuit.

According to this structure, the noise signal affecting the quality of the positioning satellite signal received from the positioning satellite can be effectively removed. Thus, the receiving capability improves.

A receiving circuit according to a third aspect of the invention includes the noise cancel type amplifying circuit described above, and an IF signal generating circuit connected with the third pin of the noise cancel type amplifying circuit to receive the addition signal and generate an IF signal.

According to this structure, the noise cancel type amplifying circuit and the IF signal generating circuit are individual components. Thus, entering noise can be reduced, and the power supply and the ground voltage can be separated. Accordingly, highly accurate noise reduction can be achieved.

An electronic device according to a fourth aspect of the invention includes the receiving circuit described above.

According to this structure, noise superimposed on a satellite signal transmitted from a positioning satellite can be reduced.

### BRIEF DESCRIPTION OF THE DRAWINGS

The invention will be described with reference to the accompanying drawings, wherein like numbers reference like elements.

FIG. 1 is a block diagram showing a structure of an electronic device according to a first embodiment.

FIG. 2 is a block diagram showing a structure of a cancel signal generating unit.

FIG. 3 shows graphs representing principle of interference wave removal.

FIG. 4 is a block diagram showing a structure of a cancel signal generating unit according to a modified example 3.

FIG. 5 is a block diagram showing a structure of an electronic device according to a modified example 4.

FIG. 6 is a block diagram showing a structure of a cancel signal generating unit according to the modified example 4.

### DESCRIPTION OF EXEMPLARY EMBODIMENTS

An electronic device according to an embodiment is hereinafter described with reference to the drawings.

### First Embodiment

### Structure of Electronic Device

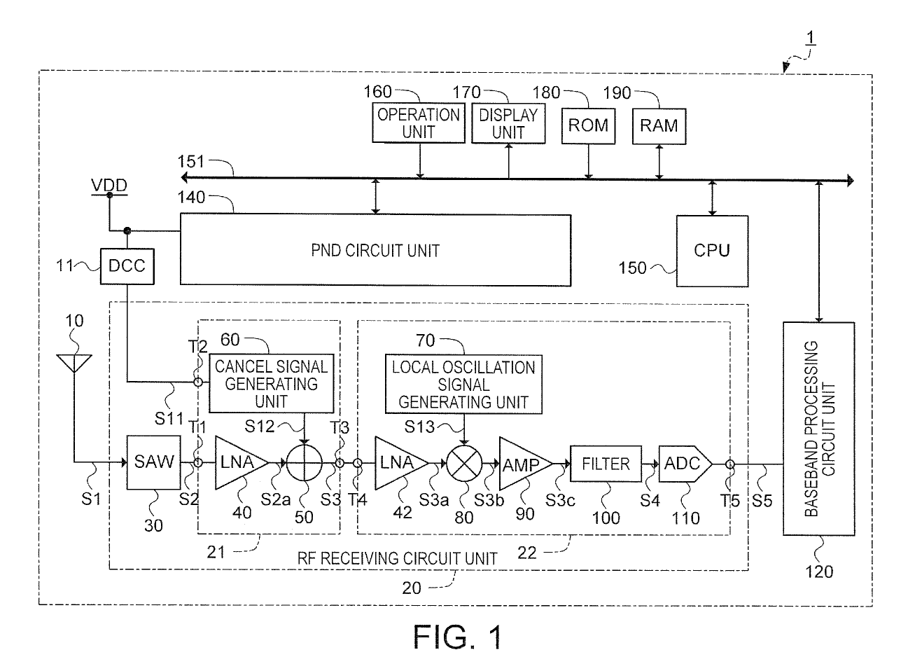

A structure of an electronic device according to a first embodiment is now described with reference to FIG. 1. FIG. 1 is a block diagram showing the electronic device in the first embodiment. According to this embodiment, a personal navigation device (hereinafter abbreviated as PND) is discussed as an example of the electronic device.

As illustrated in FIG. 1, the PND 1 includes a GPS (global positioning system) antenna 10, an RF (radio frequency) receiving circuit unit 20 as a receiving circuit, a baseband processing circuit 120, a PND circuit unit 140, a CPU (central processing unit) 150, an operation unit 160, a display unit 170, a ROM (read only memory) 180, and a RAM (random access memory) 190.

The RF receiving circuit unit 20 and the baseband processing circuit unit 120 of the PND 1 may be produced as separate LSI (large scale integration) components, or as one chip.

The GPS antenna 10 is an antenna which receives RF signals including GPS satellite signals as wireless signals transmitted from a GPS satellite as positioning satellite. The GPS antenna 10 outputs a reception signal S1 to the RF receiving circuit unit 20.

The RF receiving circuit unit 20 is an RF signal receiving circuit which has a SAW (surface acoustic wave) filter 30, a noise cancel type amplifying circuit 21, and an IF signal generating circuit 22. The receiving unit of the GPS satellite constituted by the GPS antenna 10 and the RF receiving circuit unit 20 is hereinafter referred to as GPS receiving unit.

The noise cancel type amplifying circuit 21 and the IF signal generating circuit 22 are manufactured as separate LSI components.

The noise cancel type amplifying circuit 21 includes a low-noise amplifying unit (low noise amplifier: hereinafter referred to as LNA) 40, an adding unit 50, and a cancel signal generating unit 60. The noise cancel type amplifying circuit 21 has a first pin T1, a second pin T2, and a third pin T3. The first pin T1 is connected with the input pin of the LNA 40, and the second pin T2 is connected with the input pin of the cancel signal generating unit 60. The third pin T3 is connected with the output pin of the adding unit 50.

The IF signal generating circuit 22 includes an LNA 42, a local oscillation signal generating unit 70, a multiplying unit 80, an amplifying unit (amplifier: hereinafter referred to as AMP) 90, a filter 100, and an analog-digital converting unit (analog to digital converter: hereinafter referred to as ADC) 110. The IF signal generating circuit 22 has a fourth pin T4 and a fifth pin T5.

The SAW filter 30 is a band-pass filter which passes only predetermined frequency band component of the signal S1 outputted from the GPS antenna 10 as a signal S2. The SAW filter 30 outputs the signal S2 having passed through the SAW filter 30 to the LNA 40 via the first pin T1.

The LNA 40 is a low noise amplifier which amplifies the signal S2 having passed through the SAW filter 30 to produce an amplification signal S2a. The LNA 40 outputs the amplification signal S2a to the adding unit 50.

The adding unit 50 is constituted by an adder which adds the signal S2a amplified by the LNA 40 and a cancel signal S12 produced by the cancel signal generating unit 60. The adding unit 50 outputs an addition signal S3 from the third pin T3 to the LNA 42 via the fourth pin T4. Then, the LNA 42 outputs a signal S3a produced by amplifying the addition signal S3 to the multiplying unit 80.

The cancel signal generating unit 60 inputs a noise signal S11 as alternating current component produced by cutting direct current component of power supply noise generated from a power supply VDD of the PND circuit unit 140 constituting the PND 1 by using a direct current cut (hereinafter abbreviated as DCC) 11 via the second pin T2 for phase shift. Then, the cancel signal generating signal 60 attenuates the amplitude and produces the cancel signal S12.

Since the PND circuit unit 140 is contained, the PND circuit unit 140 is disposed in the vicinity of the GPS antenna 10 or the RF receiving circuit unit 20. Thus, alternating current signals generated due to changes of electromagnetic field caused by the circuit operation of the PND circuit unit 140 are superimposed on the reception signals received by the GPS antenna 10 as interference waves. Particularly, noise at a frequency close to 1.5 GHz band as the frequency of the GPS satellite signals is superimposed as interference waves.

The local oscillation signal generating unit 70 is a circuit unit constituted by an oscillator such as LO (local oscillator) to generate a local oscillation signal S13 as multiplication RF signal. The local oscillation signal generating circuit 70 outputs the generated local oscillation signal S13 to the multiplying unit 80.

The multiplying unit 80 multiplies the signal S3a as a signal produced by amplifying the addition signal S3 from the adding unit 50 using the LNA 42 by the local oscillation signal S13 generated by the local oscillation signal generating unit 70, and outputs an IF (intermediate frequency) signal S3b produced by down-converting the RF signal into an intermediate frequency signal (IF) signal to the AMP 90.

The AMP 90 is an amplifier which amplifies the IF signal S3b outputted from the multiplying unit 80 at a predetermined amplification rate. The AMP 90 outputs the amplification signal S3c thus amplified to the filter 100.

The filter 100 is a band-pass filter which passes only components in a predetermined frequency band containing the frequency band of the signal component of the IF signal S3b contained in the signal S3c amplified by the AMP 90. The filter 100 outputs the signal S4 having passed through the filter 100 to the ADC 110.

The ADC 110 is an analog-digital converter which converts analog signals to digital signals. The ADC 110 converts the signal S4 having passed the filter 100 into the digital signal S5, and then outputs the digital signal S5 to the baseband processing circuit unit 120 via the fifth pin T5.

The baseband processing circuit unit 120 performs correlation detection process or the like for the digital signal S5 outputted from the RF receiving circuit unit 20 to capture and extract the GPS satellite signals. Then, the baseband processing circuit unit 120 decodes data and extracts aeronautical message, time information and the like to perform pseudo distance calculation, positioning calculation, and other processing. The GPS satellite signals are spectrum-spread-modulated signals called C/A (coarse and acquisition) codes.

The CPU 150 is a processor which collectively controls the respective sections of the PND 1 according to various programs such as system program stored in the ROM 180. The CPU 150 commands the display unit 170 to display navigation screen where the current position of the PND 1 chiefly positioned by the baseband processing circuit unit 120 is plotted.

The operation unit 160 is an input device constituted by operation keys, button switches and the like and outputs push signals inputted therethrough to the CPU 150. Various command inputs such as requirements for displaying navigation screen are inputted through operation of the operation unit 160.

The display unit 170 is constituted by a liquid crystal display (LCD) as a display device for providing various displays based on display signals inputted from the CPU 150. The display unit 170 displays information such as date and time, the navigation screen and others.

The ROM 180 is a memory unit for read only. The ROM 180 stores various types of programs and data such as system program for collectively controlling the PND 1 and program for providing the navigation function. The CPU 150 performs processing according to these programs and data.

The RAM 190 is a readable and writable memory unit, and forms a work area for temporarily storing the system program executed by the CPU, various types of processing program, data currently processed, process results, and the like.

An SAW filter identical to the SAW filter 30 may be inserted between the DCC 11 and the cancel signal generating unit 60. This SAW filter prevents addition of out-band noise of the signal S1 removed by the SAW filter 30 to the adding unit 50.

### Structure of Cancel Signal Generating Unit

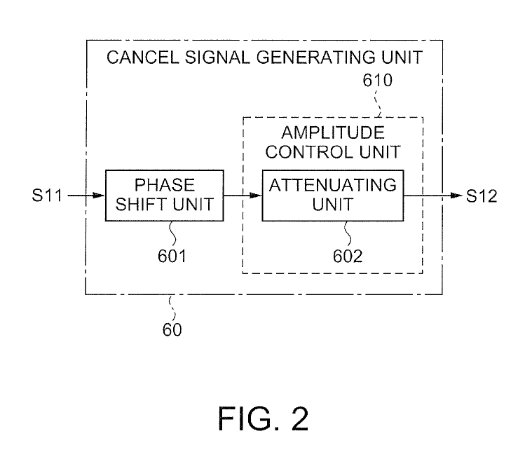

The structure of the cancel signal generating unit is now described with reference to FIG. 2. FIG. 2 is a block diagram showing the structure of the cancel signal generating unit. As illustrated in FIG. 2, the cancel signal generating unit 60 includes a phase shift unit 601 and an amplitude control unit 610 having attenuating unit 602.

The phase shift unit 601 is a phase shift circuit which arbitrarily shifts the phase of the noise signal S11 and outputs the noise signal S11 to the attenuating unit 602. The phase shift target frequency is equal to the frequency of the GPS satellite signal.

The attenuating unit 602 is an attenuator which attenuates the phase signal outputted from the phase shift unit 601 at a predetermined attenuation rate (gain). The attenuating unit 602 outputs the attenuated signal to the adding unit 50 as the cancel signal S12.

The attenuation rate of the attenuating unit 602 is established during the manufacturing step of the PND 1 after the positional relation between the GPS receiving unit and a portable electronic device is determined. More specifically, the attenuation rate is controlled such that the noise component in the band of the GPS satellite signal contained in the addition signal S3 after addition by the adding unit 50 becomes the minimum, and the control result is established as a design value.

### Principle of Interference Wave Removal

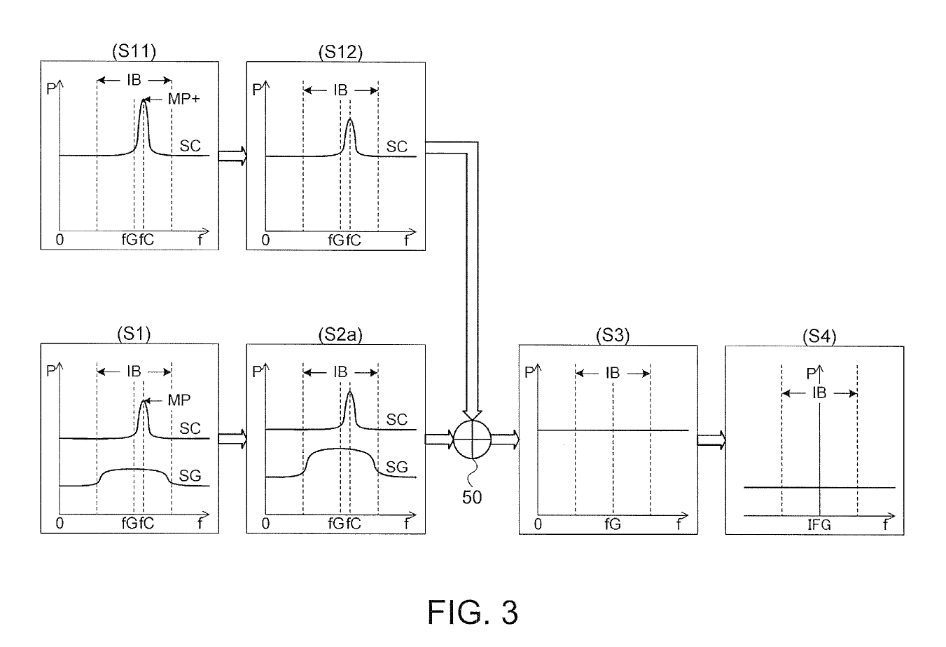

The principle of interference wave removal is now explained with reference to FIG. 3. FIG. 3 shows graphs representing the principle of interference wave removal.

FIG. 3 schematically illustrates frequency spectrums of the signals S1, S2a, S3, S4, S11, and S12 shown in FIG. 1. In these graphs, the horizontal axis indicates frequency (f), and the vertical axis indicates signal level (P). This figure shows enlarged frequency spectrums in the in-band IB area of the GPS satellite signals for simplifying the explanation in this embodiment.

The signal S1 outputted from the GPS antenna 10 to the RF receiving circuit unit 20 is the GPS satellite signal on which various types of noise are superimposed. However, most of the noises are unnecessary radiations from the electronic circuit traveling from the PND circuit unit 140 disposed in the vicinity of the RF receiving circuit unit 20 toward the GPS receiving unit. The frequency spectrum SG of the GPS satellite signal and the frequency spectrum SC of the unnecessary radiation are shown in (S1) and (S2a) in FIG. 3.

The GPS satellite signal is a signal in 1.5 GHz band where carrier waves of 1.57542 GHz (hereinafter referred to as “GPS frequency”) are spread. The GPS satellite signal has low intensity, and a predetermined band around the GPS frequency fG is the in-band IB in the frequency spectrum SG.

As for the frequency of the unnecessary radiation, the unnecessary radiation in the in-band IB is considered as noise though this depends on the respective types of digital IC. The unnecessary radiation is a signal having higher intensity than that of the GPS satellite signal, and its frequency spectrum SC has the maximum peak MP at fC (>fG) within the GPS frequency band, for example.

The unnecessary radiation signal within the GPS band becomes interference waves in the in-band IB of the received GPS satellite signal.

When the signal S1 is outputted from the GPS antenna 10 to the RF receiving circuit unit 20, the SAW filter 30 passes signal in the fixed band around the in-band IB of the GPS satellite signal contained in the signal S1. That is, the SAW filter 30 extracts signal approximately around the center of the in-band IB. Then, the LNA 40 outputs the signal S2a as the signal produced by amplifying the signal S2 having passed through the SAW filter 30 to the adding unit 50.

The frequency spectrum SC of the noise signal S11 shown in (S11) in FIG. 3 to be inputted from the DCC 11 to the cancel signal generating unit 60 is identical to the frequency component of the unnecessary radiation superimposed on the signal S1. However, the signal level MP+ is higher than that of the signal level MP of the frequency spectrum SC of the unnecessary radiation of the signal S1. The phase shift unit 601 of the cancel signal generating unit 60 shifts the phase of the inputted noise signal S11 to that opposite to the noise component of the signal S2a outputted from the LNA 40 by 180 degrees. Then, the attenuating unit 602 attenuates the noise signal S11, and outputs the resultant signal as the cancel signal S12 shown in (S12) in FIG. 3 to the adding unit 50.

Then, the adding unit 50 adds the cancel signal S12 produced by the cancel signal generating unit 60 to the signal S2a amplified by the LNA 40, amplifies the addition signal S3 shown in (S3) in FIG. 3 by the LNA 42, and outputs the amplification signal S3a to the multiplying unit 80. As a result, the shape of the frequency spectrum of the adding signal S3 has no peak on the noise contained in the in-band IB of the GPS satellite signal.

The noise peak disappears because the cancel signal S12 having phase opposite to that of the signal S2 is added to the signal S2 to cancel the signal S2. As a result, the interference waves of the digital noise superimposed on the signal S1 in the in-band IB are removed.

Then, the multiplying unit 80 multiplies the signal S3a produced by amplifying the addition signal S3 outputted from the adding unit 50 using the LNA 42 by the local oscillation signal S13 produced by the local oscillation signal generating unit 70 to down-convert the addition signal S3 and output the IF signal S3b as an IF signal to the AMP 90.

The AMP 90 receives the IF signal S3b outputted from the multiplying unit 80 and amplifies the IF signal S3b, and then outputs the resultant signal S3c to the filter 100. The filter 100 passes signal in the band corresponding to the intermediate frequency of the GPS satellite signal (band range of intermediate frequency corresponding to in-band IB) contained in the signal S3c amplified by the AMP 90, and outputs the resultant signal as the signal S4 shown in (S4) in FIG. 3 to the ADC 110.

The signal S4 is a signal contained in the out-band signal and shielded by the filter 100 as shown in (S4) in FIG. 3. Since the signal S4 is an IF signal, the frequency spectrum is shifted to the low band side as a whole. In this case, the center frequency IFG of the IF signal, that is, the center frequency of the in-band IB corresponds to the vertical axis.

According to this embodiment, the following advantages are offered.

In this embodiment, the signal received by the GPS antenna 10 is amplified by the LNA 40, and then the cancel signal produced by the cancel signal generating unit 60 is added by the adding unit 50. As a result, the noise in the in-band superimposed on the reception signal is cancelled.

Thus, the reception signal is amplified by the LNA 40, and the cancel signal is added to the reception signal after the signal level is raised. In this case, level control of the cancel signal is easier than in the case of addition of the cancel signal immediately after the GPS antenna 10. Accordingly, increase in the interference wave component caused by the excessive cancel signal can be prevented.

While the electronic device according to this embodiment has been described herein, various changes and modifications may be made without departing from the scope and spirit of the invention. The followings are examples of such modification.

### MODIFIED EXAMPLE 1

An electronic device according to a modified example 1 is now described. While the PND 1 as an example of electronic device has been discussed in the first embodiment, the invention is applicable to various types of electronic device such as cellular phone, car navigation device, and personal computer (PC). That is, the invention is applicable to any types of electronic device which includes a receiving circuit receiving wireless signals and an electronic device generating signals as interference waves (noise) for the reception signals.

The receiving circuit may be various types of circuit such as communication circuit as well as the GPS signal receiving circuit. The electronic circuit generating interference waves for the reception signals from the receiving circuit may be a computer system, various types of communication circuit or the like.

### MODIFIED EXAMPLE 2

An electronic device according to a modified example 2 is now explained. While the GPS has been discussed as an example of the satellite positioning system according to the first embodiment, the invention is applicable to various types of satellite positioning system such as WAAS, QZSS, GLONASS, and GALILEO.

### MODIFIED EXAMPLE 3

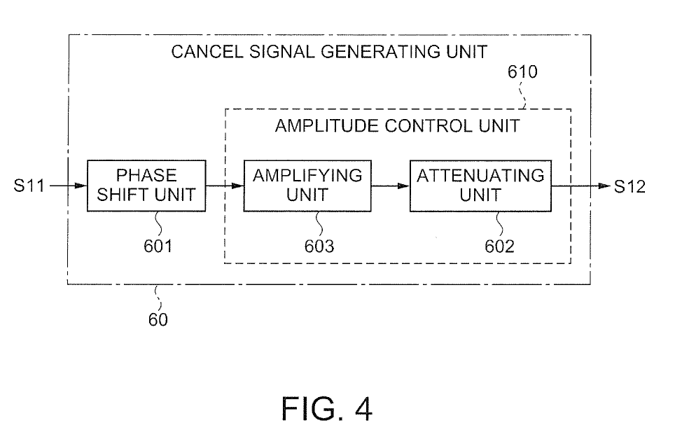

An electronic device according to a modified example 3 is now described. While the cancel signal generating unit 60 according to the first embodiment has the attenuating unit 602, an amplifying unit 603 may be provided as illustrated in FIG. 4. In this structure, the amplitude of the phase signal outputted from the phase shift unit 601 can be either amplified or attenuated.

### MODIFIED EXAMPLE 4

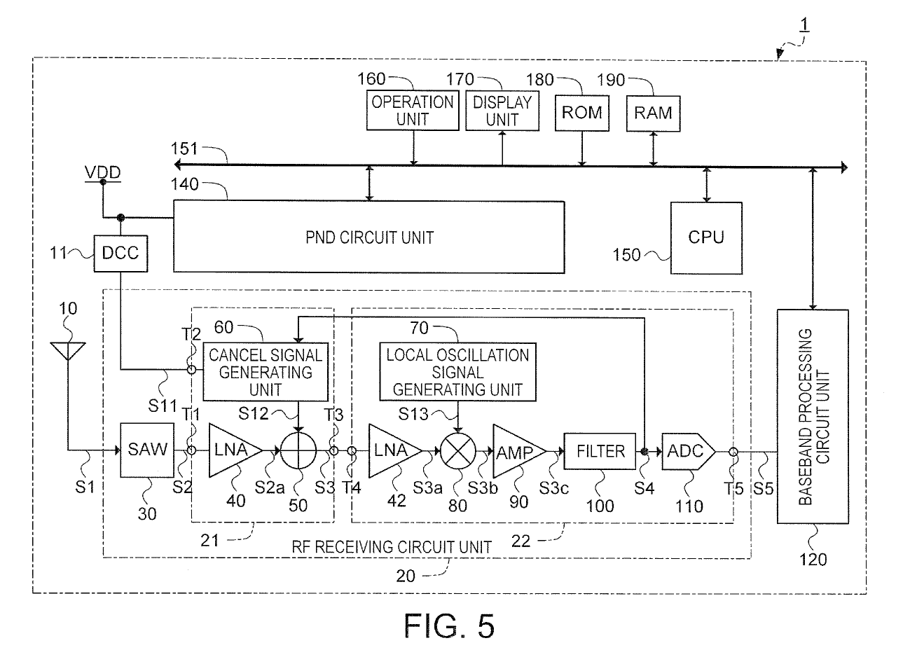

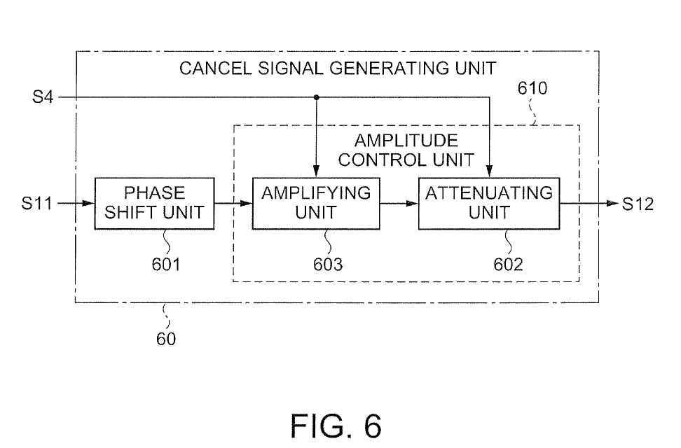

An electronic device according to a modified example 4 is now explained. The cancel signal generating unit 60 may be a unit capable of controlling the attenuation rate of the attenuating unit 602 based on the signal obtained after addition by the adding unit 50. It is expected that the electronic circuit disposed in the vicinity of the GPS receiving unit does not always perform constant circuit operation, but appropriately changes the circuit operation at the time of operation or stop or in other conditions. FIG. 5 is a block diagram showing the structure of the PND 1 according to the modified example 4. As can be seen from FIG. 5, the signal S4 outputted from the filter 100 is fed back to the cancel signal generating unit 60 as a signal after addition by the adding unit 50. FIG. 6 is a block diagram showing the structure of the cancel signal generating unit 60 according to the modified example 4. The amplification rate of the amplifying unit 603 and the attenuation rate of the attenuating unit 602 are determined based on the signal S4. While the signal S4 is inputted to both the amplifying unit 603 and the attenuating unit 602 in FIG. 6, the signal S4 may be inputted to either of those.

Japanese Patent Applications No. 2007-248582 filed on Sep. 26, 2007 and No. 2008-174258 filed on Jul. 3, 2008 are hereby incorporated by reference in its entirety.

What is claimed is:

1. A noise cancel method comprising:
generating a cancel signal which cancels a noise signal based on the noise signal generated from a receiving unit for receiving a wireless signal;
generating an amplification signal produced by amplifying a reception signal inputted to the receiving unit; and
generating an addition signal produced by adding the amplification signal and the cancel signal.

2. The noise cancel method according to claim 1, further comprising performing amplitude control of the noise signal based on the addition signal at the time of generation of the cancel signal.

3. The noise cancel method according to claim 1, further comprising performing amplification or attenuation of the noise signal based on the addition signal at the time of generation of the cancel signal.

4. The noise cancel method according to claim 1, wherein the reception signal is a positioning satellite signal received from a positioning satellite.

5. A noise cancel type amplifying circuit, comprising:
a first pin through which a reception signal is inputted;
a second pin through which a noise signal affecting the reception signal is inputted;
a low-noise amplifying circuit connected with the first pin to input the reception signal and output an amplification signal;
a cancel signal generating unit connected with the second pin to generate a cancel signal for canceling the noise signal based on the noise signal inputted through the second pin; and
an adder which outputs an addition signal produced by adding the amplification signal and the cancel signal through a third pin.

6. The noise cancel type amplifying circuit according to claim 5, wherein the cancel signal generating unit has a phase shift unit for outputting a phase shift signal produced by arbitrarily shifting the phase of the noise signal, and an amplitude control unit for controlling the amplitude of the phase shift signal.

7. The noise cancel type amplifying circuit according to claim G, wherein the amplitude control unit includes at least either an amplifying unit or an attenuating unit.

8. The noise cancel type amplifying circuit according to claim 7, wherein at least either the amplification rate of the amplifying unit or the attenuation rate of the attenuating unit is determined based on the addition signal.

9. The noise cancel type amplifying circuit according to claim 5, wherein:
the reception signal is a positioning satellite signal received from a positioning satellite; and
the noise signal is a signal generated from a circuit disposed in the vicinity of a receiving unit for receiving the receiving signal.

10. A receiving circuit, comprising:
the noise cancel type amplifying circuit according to claim 5; and
an IF signal generating circuit connected with the third pin of the noise cancel type amplifying circuit to receive the addition signal and generate an IF signal.

11. An electronic device, comprising the receiving circuit according to claim 10.
